(19) United States
(12) Patent Application Publication
(10) Pub. No.: US 2009/0224004 A1
(43) Pub. Date: Sep. 10, 2009

(54) APPLICATOR WITH AN EXCHANGEABLE CONTAINER

(75) Inventors: Uwe Müller, Köln (DE); Venkata-Rangarao Kanikanti, Leverkusen (DE); Gerald Beddies, Overland Park, KS (US); Joy Olsen, Köln (DE); Ernst Böttcher, Köln (DE); Georg Schulte, Wuppertal (DE); Bernhard Saalfeld, Köln (DE); Nikolaus Kowollik, Merzenich-Girbelsrath (DE); Herbert Argauer, Pirk (DE); Michael Kloiber, Pfreimd (DE)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/297,586

(22) PCT Filed: Apr. 10, 2007
(86) PCT No.: PCT/EP07/03166

§ 371 Date: Oct. 17, 2008

(30) Foreign Application Priority Data
Apr. 22, 2006 (DE) 102006018827.6

(51) Int. Cl. G01F 11/02; B05C 17/01; B65D 83/76; A61C 5/06

(52) U.S. Cl. 222/309; 222/391; 433/89; 222/326

(57) ABSTRACT

The invention relates to an applicator for the application of liquid or pasty media, containing a housing for receiving a dosing unit and an adapter for fastening a container, for the medium to be applied, to the applicator; a dose setting means; a lever for the application of the apportioned amount, the lever interacting mechanically with the dosing unit and with the dose setting means. The lever is mounted moveably at its proximal end within the housing. The distal end of the lever spreads apart from the housing, with the distance of the distal end of the lever from the housing being dependent on the dose set.

The invention relates to an applicator and an exchangeable container containing a liquid or pasty medium, which container can be fastened to the applicator, for oral application of liquid or pasty media, in particular to an animal.

Applicators for liquid or pasty media are known for a very wide variety of applications. DE 103 48 186 A1 discloses an injection device for administering a fluid product, for example a medicine, for the treatment of osteoporosis. DE 100 20 591 A1 relates to an applicator for dental material, which applicator has a high degree of dosing accuracy because of a click-and-pull mechanism and a blocking mechanism and is free from secondary flow phenomena. EP 0 806 187 B1 likewise relates to an applicator in the dental sphere, which is intended to avoid repressing the compound to be applied.

With these applicators known from the prior art, it is not possible to set different doses. They are therefore not suitable for the application of different doses as may be required, for example, in the treatment of animals if a dose dependent on the particular weight of the animal is to be administered. The object according to the invention is therefore to provide an easy-to-operate applicator for the application of liquid or pasty media. The applicator is to be suitable, in particular, for applying different dosing volumes selected by the user.

The solution according to the invention resides in an applicator for the application of liquid or pasty media, containing a housing for receiving a dosing unit and an adapter for fastening a container, for the medium to be applied, to the applicator, a dose setting means, a lever for the application of the apportioned amount, which lever interacts mechanically with the dosing unit and with the dose setting means, the lever being mounted moveably at its proximal end within the housing and the distal end of the lever spreading apart from the housing in the state ready for application, with the distance of the distal end of the lever from the housing being dependent on the dose set.

In the vicinity of its proximal end mounted in the housing, the lever has a ball which is in contact with the dosing unit. The dosing unit contains a threaded spindle, a threaded sleeve, a pressure-exerting means, a latching mechanism for the threaded sleeve and a latching mechanism for the threaded spindle.

At its front end, the housing has an opening for the fastening of a container, which opening is filled with the medium to be applied. The threaded spindle and the threaded sleeve are mounted coaxially in the housing in such a manner that the external thread of the threaded spindle engages in the internal thread of the threaded sleeve. The ball belonging to the lever is arranged on an axis with the threaded sleeve, threaded spindle and housing opening in such a manner that a movement of the spread-apart, distal end of the lever toward the housing causes a movement of the ball in the direction of the front, open end of the housing. In the process, the ball likewise presses the threaded sleeve with the threaded spindle mounted therein in the direction of the front, open housing end. The movement of the spread-apart, distal end of the lever toward the housing takes place counter to the pressure exerted by the pressure-exerting means such that, when the lever is released, it passes again into the spread-apart position and the ball is removed again from the front, open housing end. Threaded sleeve, ball and pressure-exerting means are mounted with respect to one another in such a manner that the threaded sleeve is pushed with the ball away from the front, open housing end when the ball is removed from the front, open housing end. The threaded spindle has a quick-acting screw thread. As a result, an axial force which acts on the threaded sleeve can be converted into a rotational movement of the threaded sleeve relative to the threaded spindle. When a cartridge is inserted, the latching mechanism for the threaded sleeve permits only a displacement of the threaded sleeve relative to the threaded spindle in the axial direction to the rear end of the housing but not in the opposite direction. When the cartridge is inserted, the latching mechanism for the threaded spindle permits only a displacement of the threaded spindle in the axial direction to the front end of the housing, but not in the opposite direction, and therefore the threaded spindle does not follow the threaded sleeve during the movement toward the rear housing end. Thus, after each lever pressure, the threaded spindle is pushed a section further out of the front end of the threaded sleeve. The latching mechanism for the threaded spindle is connected to the adapter for the cartridge in such a manner that, when the cartridge is removed from the adapter, the latching mechanism for the threaded spindle is simultaneously released and any desired displacement of the threaded spindle in the axial direction relative to the threaded sleeve and to the housing is possible. The length of the threaded spindle is dimensioned in such a manner that, during each application, its front end is moved somewhat further out of the front housing opening. If a container for receiving the medium to be applied is fastened to the adapter at the front end of the housing, then the threaded spindle is pushed further into said container with each application, i.e. with each lever pressure.

The dose setting means determines the degree of spreading apart of the lever before an application and therefore the stroke toward the front housing end that is executed by the ball, the threaded sleeve and therefore the threaded spindle. The dose setting means can be a dosing wheel which is fitted on that side of the ball which faces away from the front housing end on an axis with the ball and the front housing opening and delimits the axial movement clearance of the ball. The dosing wheel may also be connected to the threaded sleeve. A scale for the dose can be fitted to the distal end of the lever in such a manner that a larger or smaller part of the scale is visible, depending on the degree of spreading apart of the lever. However, a scale may alternatively also be fitted directly to the dosing wheel or at another suitable location, for example on the outside of the housing. In this case, the lever may be designed in such a manner that, in the bearing position of the lever, the scale is concealed, and, when the lever is pivoted out, the scale is visible, in accordance with the apportioned amount.

The lever preferably has a child-proof lock on the housing. The child-proof lock can consist, for example, in that the lever, which initially bears against the housing after an application, can be brought again into the spread-apart position only after a latch-in position of the lever on the housing is overcome.

In a further embodiment of the invention, a transmission sleeve with a thread internally and a thread acting in the same direction externally is mounted concentrically between the threaded sleeve and the threaded spindle. The internal thread of the transmission sleeve has a different pitch from the internal thread of the threaded sleeve. The advance of the threaded spindle changes in accordance with the ratio between pitch of the internal thread of the threaded sleeve and pitch of the internal thread of the transmission sleeve. Therefore, when the threaded sleeve is rotated back, the transmission sleeve does not execute a return stroke, and rotation with the threaded sleeve is blocked by a latching element.

Various embodiments are possible for the adapter for the fastening of a container containing the medium to be applied. Since the container containing the medium to be applied interacts with the adapter, the container, for example a cartridge, is matched to a certain adapter. The interaction of a specially adapted cartridge and adapter is intended to prevent random, unsuitable cartridges being fastened to the adapter, since otherwise wrong or inaccurate doses could occur. The interaction of adapter and container is explained using a cartridge as representative of other possible containers.

The cartridge according to the invention, which can be filled or is filled with a liquid or pasty medium, preferably with an animal drug which is to be applied, has a cylindrical housing with a filling opening on the one side and with a tapered region, the mouthpiece, on the other side of the housing. In the case of a filled cartridge, the filling opening is closed by a plunger which, upon application, is displaced successively further into the interior of the cartridge toward the mouthpiece. The mouthpiece has an opening for dispensing the liquid or pasty medium from the interior of the cartridge. The cartridge typically has a volume within the range of from 5 ml to 50 ml, preferably within the range of from 8 ml to 20 ml, particularly preferably from 11 to 13 ml.

A cartridge ready for use can be brought with its side closed by the plunger to the adapter in the interior of the housing through the opening at the front end of the housing of the applicator, the passage opening. The passage opening is circular, but has one or more additional openings, as a result of which the diameter of the circular shape is partially increased. The cartridge, which is adapted to a passage opening of this type, correspondingly has, in the region about its circular filling opening, extension pieces which are inverse to the additional openings. The passage opening preferably has two to five, particularly preferably two mutually opposite additional openings. The cartridge correspondingly has two to five, particularly preferably two mutually opposite extension pieces.

The adapter is located behind the passage opening. In a first embodiment, the adapter has a lock nut and a pressure-exerting means, for example a compression spring. The lock nut is a tubular sleeve which is mounted displaceably in the axial direction concentrically with the threaded spindle and directly behind the passage opening.

The pressure-exerting means presses the lock nut in the direction of the passage opening at the front end of the housing.

The cartridge is fastened to the adapter by being guided with its side closed by the plunger to the passage opening such that the extension pieces enter through the additional openings into the interior of the applicator housing. In the process, axial pressure is exerted on the pressure-exerting means in the direction of the rear end of the housing and at the same time the cartridge is rotated in such a manner that the extension pieces come to lie behind the circular regions of the passage opening on the other side of the additional openings where they are clamped or latched in place.

The lock nut furthermore has the function in the state ready for use, i.e. when a cartridge is fastened to the adapter, of producing an interlocking connection between the threaded sleeve or the transmission sleeve and the threaded spindle. This takes place by the front end of the threaded sleeve or the transmission sleeve only lying within the tubular sleeve of the lock nut when a cartridge is fastened to the adapter. If a cartridge is not located on the adapter, then the lock nut is displaced by the pressure-exerting-means closure toward the front end of the housing until the front end of the threaded sleeve or of the transmission sleeve is no longer located within the tubular sleeve of the lock nut. The threaded sleeve or the transmission sleeve is slit longitudinally such that it spreads as soon as its front end is no longer held together by the tubular part of the lock nut. This spreading is sufficient in order to undo the close contact between threaded spindle and threaded sleeve or transmission sleeve such that the threaded spindle can then be moved in the axial direction by the threaded sleeve or transmission sleeve without carrying along the threaded sleeve or transmission sleeve. This axial possibility of moving the threaded spindle relative to the threaded sleeve or transmission sleeve is necessary so that, after a used cartridge is removed, the threaded spindle can be pushed back into the housing before a new unused cartridge is fitted.

In a second embodiment, the adapter has a lock nut element. The lock nut element is a two-part, tubular sleeve which is mounted rotatably concentrically with the threaded spindle and with the threaded sleeve or transmission sleeve and displaceably in the axial direction. On its side facing the passage opening, it has, on its outer extent, one or more grooves or projections which extend in the axial direction and to which a correspondingly adapted cartridge can be coupled. One to eight, particularly preferably two mutually opposite grooves or projections are preferred. On its side facing away from the passage opening, the cartridge has, on its outer circumference, at least one cam which engages in at least one channel which is provided on the inner wall of the housing. The one or more channels run on the inner wall of the housing at an angle within the range of between 90 and 100 degrees, preferably between 90.5 and 95 degrees, with respect to the axial direction. Upon rotation of the lock nut element, the lock nut element is displaced simultaneously in the axial direction, guided by the cam or the cams in the channel.

A bushing with a central hole through which the threaded spindle passes is located centrally in the interior of the lock nut element. Upon axial displacement of the lock nut element, the bush is pushed in a direction away from the passage opening of the housing, over that end of the threaded sleeve or transmission sleeve which faces the passage opening. Since the threaded sleeve or transmission sleeve is of elastic design because of slits and spreads, the displacement of the bushing over the end of the threaded sleeve or transmission sleeve produces the interlocking connection between the threaded spindle and the threaded sleeve or transmission sleeve. Furthermore, the bushing prevents the occurrence of lateral forces which would obstruct the rotational movement of the threaded sleeve.

The cartridge, which is adapted for the second embodiment of the adapter, has, in its inner circumference, in the region of the filling opening, one or more elongate grooves or projections extending in the axial direction. One to eight, particularly preferably two mutually opposite grooves or projections are preferred. The position of these elongate grooves or projections is selected in such a manner that they coincide with the position of the grooves or projections in the outer circumference of the lock nut element, and the cartridge interacts with the lock nut element in accordance with the lock and key principle when it is pushed over the lock nut element. The cartridge therefore has projections in its inner circumference when the lock nut element has grooves on its outer circumference, and vice versa.

During fitting of the cartridge to the adapter according to the second embodiment, it is rotated in such a manner that the positions of the extension pieces coincide with the position of the additional openings in the passage opening. This means that the position of the grooves or projections on the lock nut element and the grooves or projections on the inner wall of the cartridge simultaneously coincide. Thus positioned, the cartridge is pushed over the lock nut element. During a rotation of the cartridge, the lock nut element executes an axial movement relative to the housing via the interaction of cam and channel. During this movement, the threaded sleeve or transmission sleeve is enclosed and the interlocking connection with the thread of the threaded spindle produced. In their final position, the extension pieces latch into depressions or apertures on the inner wall of the housing.

In a further embodiment, the applicator according to the invention also has additional locking of the cartridge in order to prevent strong rotational forces, which act on the cartridge, for example when unscrewing a closure cap (see below) from the mouthpiece of the cartridge, from resulting in the cartridge being released from the adapter. The locking of the cartridge consists in an approximately semicircular latching arc. The latching arc is mounted concentrically in the housing in the region of the adapter and has latching cams which latch to corresponding latching cams on the extension pieces of the cartridge. Furthermore, the latching arc has two sensing devices arranged opposite each other on the ends of the arc. The arcuate shape results in the latching arc being elastic and being able to deform by radial pressure from the outside on the sensing devices in such a manner that the sensing devices come closer to each other. After the sensing devices are released, the latching arc resumes its original shape. In the region of the sensing devices, sawtooth-shaped latching cams are arranged on the inside of the latching arc and both point with their tips in the same rotational direction.

The latching arc is arranged in the housing of the applicator in such a manner that the sensing devices come to lie in apertures through the housing and can be operated, i.e. pressed, from outside the housing. If a cartridge is in the latched state, sawtooth-shaped latching cams on the extension pieces of the cartridge engage in the sawtooth-shaped latching cams of the latching arc in such a manner that it is no longer possible to rotate the cartridge back. Only by pressing on both sensing devices at the same time is the latching of the respective latching cams at the two mutually opposite positions released. This takes place in that the sensing devices are compressed to an extent such that the latching cams of the latching arc are displaced into the interior of the housing until the latching cams of the cartridge can be guided on the outside past the latching cams of the latching arc.

In a preferred embodiment, the cartridge has a closure cap on the mouthpiece. This may be a rotary cap which is known per se, has an internal thread and is screwed onto the mouthpiece, which is provided with an external thread. In a preferred embodiment, the closure cap has a child-proof lock. A closure cap with a child-proof lock can be constructed as follows: the closure cap with a child-proof lock has an outer cylindrical wall and an end wall. The outside diameter of the closure cap corresponds to the outside diameter of the cartridge onto the mouthpiece of which it is to be screwable. An inner cylindrical wall is located concentrically within the outer wall. A thread is located on the inside of the inner wall. On the open side of the closure cap, two mutually opposite cams are located on the end surface of the outer wall. The mouthpiece of the cartridge has an external thread which is matched to the thread on the inside of the inner wall. In that region of the cartridge in which it tapers to the mouthpiece, the wall has an annular edge on the inside of which two mutually opposite, sawtooth-shaped projections are located. When the closure cap is screwed to the cartridge, the cams are guided past the projections and latch behind them such that it is no longer possible to rotate the closure cap back. In order to rotate it back, i.e. in order to unscrew the closure cap, the latching of the cams to the projections has to be overcome. This takes place by the outer wall of the closure cap being compressed in the region of the cams until the cams can be guided on the inside past the projections. This compression and rotation requires some expenditure of force and dexterity and is normally not possible for children. The closure cap described therefore has a child-proof lock.

On the outside of the outer wall of the closure cap, structures can be incorporated in the region of the cams, said structures making it possible for the closure cap to grip securely and hold firmly in these regions.

The invention furthermore relates to an applicator system containing an above-described applicator to which an above-described cartridge is fastened. The fastening is reversible and therefore an empty cartridge can be replaced by a new full one. Upon each lever pressure (stroke), the cartridge fastened to the applicator is emptied by a predetermined volume by, upon each lever pressure, the plunger being pushed a section further into the cartridge by the threaded spindle, which protrudes out of the housing coaxially with the cartridge. In the process, upon each lever pressure, a dosing volume preferably in the range of from, for example, 0.1 to 2.5 ml is applied, with it being possible for the dose to be varied within, for example, 0.1 ml steps. The applicator system according to the invention is particularly suitable for oral application, very particularly for oral application of a drug to animals.

An even greater range of variation of drug doses per lever pressure (stroke) can be achieved by cartridges being used with media which contain various product concentrations, for example three different concentrations of 0.03%, 0.5%, 2.4%, which, given a possible dosing volume of 0.1 to 2.5 ml, corresponds to a dosing range of 0.00009 ml to 0.06 ml.

For further variation of the dosing volumes, cartridges with a different diameter can also be used, with then, for each cartridge diameter, a dedicated scale being fitted to the lever or at another suitable location on the applicator.

A detailed illustration of the various embodiments of the invention can be gathered from the figures and from the description below.

### FIGURES AND EXAMPLES

FIG. 1 shows an exterior view of a first embodiment of the applicator with the lever pivoted out to the maximum

FIG. 2 shows an interior view of a first embodiment of the applicator with the lever bearing against it

FIGS. 3 a, b show an interior view of a first embodiment of the applicator with the lever pivoted out to the maximum, and a view of a detail

FIG. 4 shows an interior view of a first embodiment of the applicator with the lever bearing against it

FIG. 5 shows an exterior view of a second embodiment of the applicator with the lever bearing against it

FIG. 6 shows an interior view of a second embodiment of the applicator with the lever bearing against it

FIG. 7 shows an interior view of a second embodiment of the applicator with the lever pivoted out to the maximum

FIGS. 8 a, b show an interior view of a variant of the first embodiment of the applicator with the stroke-type transmission

FIG. 9 shows a cartridge

FIG. 10 shows the mouthpiece region of a cartridge

FIGS. 11 a, b show the closure cap and the mouthpiece region of a cartridge

FIG. 12 shows a slit transmission sleeve

FIG. 13 shows a variant of the adapter for the cartridge

FIG. 14 shows the locking of the cartridge for fastening the cartridge to the applicator

FIGS. 15a-c show views of details of the mechanism of the locking of the cartridge

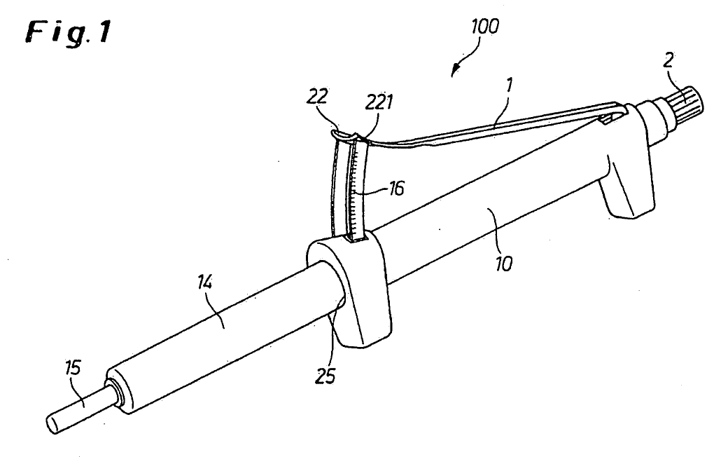

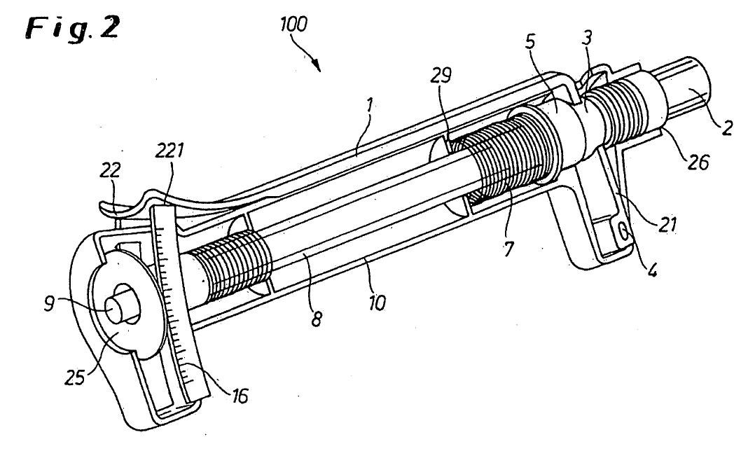

FIGS. 1 to 4 show a first embodiment of the applicator according to the invention.

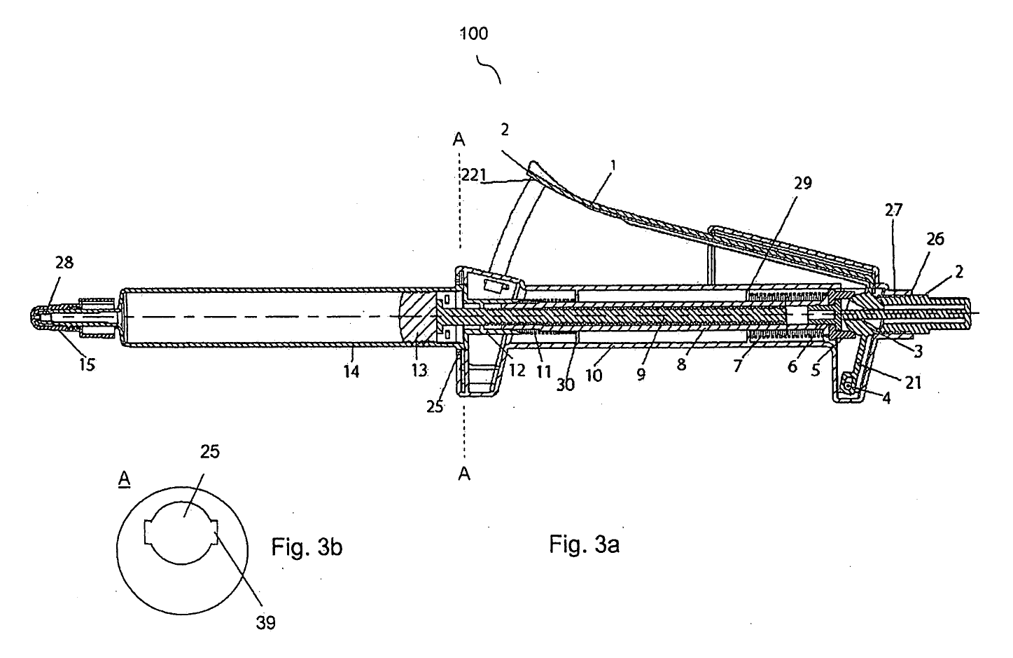

The applicator 100 has an elongate housing 10 for receiving a dosing unit. At its front end, the housing 10 has a passage opening 25 (also see FIG. 3b). An adapter for the fitting of a container, for example a cartridge 14 for the liquid or pasty medium, is located in the region of the passage opening 25.

At its rear end, the housing 10 has an opening 26 for a dosing wheel 2. In the region of the opening 26, a nut thread 27, in which the dosing wheel 2 is mounted rotatably, is fitted on the inside at the rear end of the housing 10. The dose for application is set via the dosing wheel 2.

The lever 1 is used for the application of the apportioned amount and is mounted moveably at its proximal end 21 within the housing 10. In FIG. 1, the distal end 22 of the lever 1 is spread apart from the housing 10, with the distance of the distal end 22 of the lever 1 from the housing 10 being dependent on the dose set. The lever 1 is connected rotatably to the housing 10 at a fulcrum 4.

A scale 16 is fitted to the distal end 22 of the lever 1 and is located relative to the fulcrum 4 on a sector of the circular path of a circle, the radius of which corresponds to the distance between fulcrum 4 and the point 221 at which the scale 16 is fitted to the lever 1. The scale 16 is located to a greater or lesser extent within or outside the housing 10, depending on the degree of spreading apart of the lever 1. At high doses, a higher proportion of the scale 16 is located outside the housing 10, and, correspondingly, a smaller part at low doses.

The lever 1 has a ball 3 which is in contact with the dosing wheel 2, on the one hand, and the dosing unit. The dosing unit contains a threaded spindle 9, a threaded sleeve 8, a compression-spring dosing means 7, a latching mechanism for the threaded sleeve 8 and a latching mechanism for the threaded spindle 9.

The compression-spring dosing means 7 is mounted between a ring 29, which is connected fixedly to the housing 10, and the rear end of the threaded sleeve 8 in such a manner that it exerts axial pressure on the rear end of the threaded sleeve 8 in the direction of the rear end of the housing 10.

The threaded spindle 9 has a quick-acting screw thread. As a result, an axial force which acts on the threaded sleeve 8 can be converted into a rotational movement of the threaded sleeve 8 relative to the threaded spindle 9.

When the cartridge 14 is inserted, the latching mechanism for the threaded sleeve 8 permits only a displacement of the threaded sleeve 8 relative to the threaded spindle 9 in the axial direction to the rear end of the housing 10, but not in the opposite direction. When the cartridge 14 is inserted, the latching mechanism for the threaded spindle 9 permits only a displacement of the threaded spindle 9 in the axial direction to the front end of the housing 10, but not in the opposite direction. The latching mechanism for the threaded spindle 9 is connected to the adapter for the cartridge 14 in such a manner that, when the cartridge 14 is removed from the adapter, the latching mechanism for the threaded spindle 9 is simultaneously released and any desired displacement of the threaded spindle 9 in the axial direction relative to the threaded sleeve 8 and to the housing 10 is possible.

The latching mechanism functions as follows: when axial pressure is applied to the sleeve 5, which is secured against rotation and therefore permits only a movement in the axial direction, a frictional connection is produced by the ball 3, by the friction in relation to the end surface of the threaded sleeve 6, and therefore rotation of the threaded sleeve 8 is prevented. Alternatively, a toothing which produces an interlocking connection can be provided on the end surfaces of the sleeve 5 and threaded sleeve 8. During the return stroke, the compression-spring dosing means 7 presses axially onto the sleeve 5. The interlocking connection or frictional connection is released by the axial play of the sleeve 5 between the threaded sleeve 8 and the knob 6. The threaded sleeve 8 can rotate back.

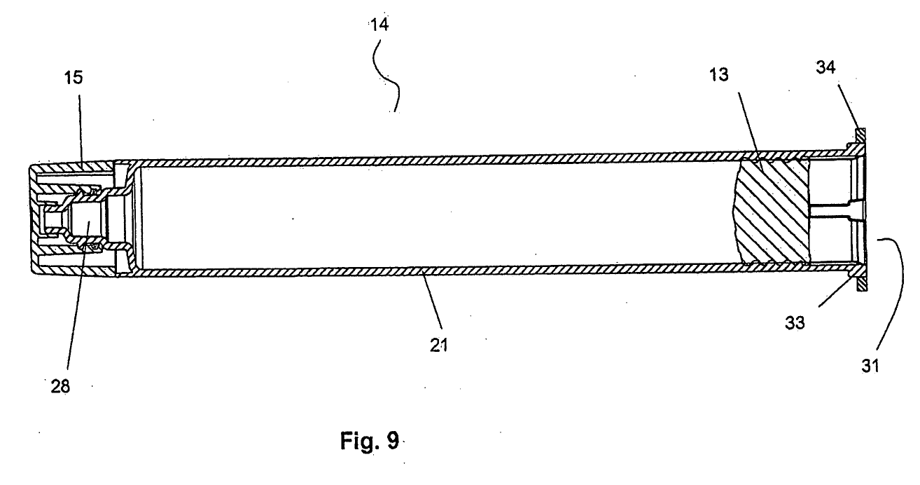

FIGS. 1, 3a and 4 show a cartridge 14 which is fastened to the front end of the housing 10 via the adapter. FIG. 9 shows an individual view of the cartridge. That end of the cartridge 14 which faces the applicator 100 has an opening 31 for filling the cartridge 14, which opening is closed by a plunger 13 in the state ready for use. The other end of the cartridge 14 tapers to form a mouthpiece 28 which is closed by a closure cap 15.

To set the dose, the dosing wheel 2 is rotated out of the housing 10 in the nut thread 27. The compression-spring dosing means 7 presses against the rear end of the threaded sleeve 8, which is in turn in contact with the sleeve 5, the knob 6 and the ball 3. With the dosing wheel 2, the ball 3 with the sleeve 5 and the knob 6 is displaced in the axial direction toward the rear end of the housing 10. By displacement of the ball 3 in the direction of the rear end of the housing 10, the lever 1 pivots outward via the fulcrum 4 and, with increasing axial displacement, spreads apart at its distal end 22 ever more from the housing 10. The dose set can be gathered from the scale 16.

Under the axial pressure of the compression-spring dosing means 7 toward the rear end of the housing, the threaded sleeve 8 is screwed to the rear on the thread of the threaded spindle 9. In this case, the threaded sleeve 8 is mounted rotatably via the sleeve 5 and knob 6. The threaded spindle 9 is prevented the means for securing the threaded spindle 9 against rotation from rotating with the threaded sleeve 8. Only the threaded sleeve 8 is therefore pushed axially to the rear.

For the application, the distal end 22 of the lever 1 is pressed in the direction of the housing 10. In the process, the ball 3 is displaced in the axial direction counter to the pressure of the compression-spring dosing means 7 to the front end of the housing 10. With the ball 3, the sleeve 5, the knob 6 and the threaded sleeve 8 are moved axially in the direction of the front end of the housing 10. On account of the latching mechanism for the threaded sleeve 8, the threaded spindle 9 is displaced axially with the threaded sleeve 8 in the direction of the front end of the housing. The latching mechanism for the threaded sleeve 8 does not permit any relative rotation between threaded spindle 9 and threaded sleeve 8 in this direction while the guide for the threaded spindle 9 permits a movement in the direction of the front end of the housing 10.

The threaded spindle 9 presses against the plunger 13 of the cartridge 14 which is fastened to the adapter at the front end of the housing 10. The plunger 13 is therefore moved into the cartridge 14 and, for its part, presses the amount of medium corresponding to the dose set out of the cartridge 14 through the mouthpiece 28.

After the lever 1 is released, it pivots out again under the pressure of the compression-spring dosing means 7 in accordance with the dosing volume previously set by means of the dosing wheel 2. In this case, as described above, the threaded sleeve 8 is screwed further to the rear on the thread of the threaded spindle 9 by the axial force of the compression-spring dosing means 7. The threaded spindle 9 is prevented by the means of securing the threaded spindle 9 from rotation from being screwed axially to the rear with the threaded sleeve 8.

The application is made either as described above again, or a smaller or larger dosing volume can be set via the dosing wheel 2. The fact that the setting of a smaller volume does not trigger the applicator can be explained as follows:

the dosing wheel 2 has a cylinder which, when a smaller volume is set, presses by means of the ball 3 onto the knob 6. This pressure causes the knob 6 to be displaced forward with the threaded sleeve 8. The interlocking or frictional connection is released. The threaded sleeve 8 is screwed forward without carrying out a stroke on the threaded spindle 9.

After a first dose and every further dose is set, upon every actuation of the lever 1 the threaded spindle 9 is pushed further in the axial direction out of the front end of the housing 10.

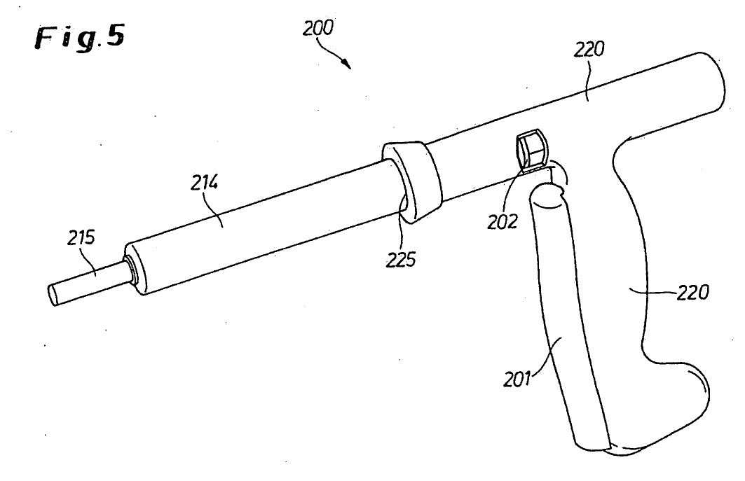

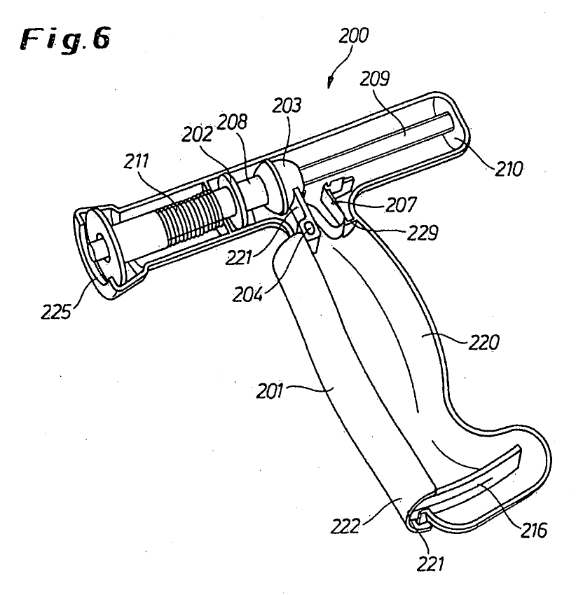

FIGS. 5 to 7 show a second embodiment of the applicator according to the invention.

The applicator 200 has an elongate housing part 210 for receiving a dosing unit and a housing part 220 which protrudes from the elongate housing part 210 in the central region thereof and is shaped in the form of a handle. At its front end, the housing part 210 has a passage opening 225 and, in the region of the passage opening 225, an adapter for fitting a container, for example a cartridge 214 for the liquid or pasty medium.

A dosing wheel 202 is mounted rotatably within the housing part 210 via the threaded spindle 209. The dose for the application is set via the dosing wheel 202.

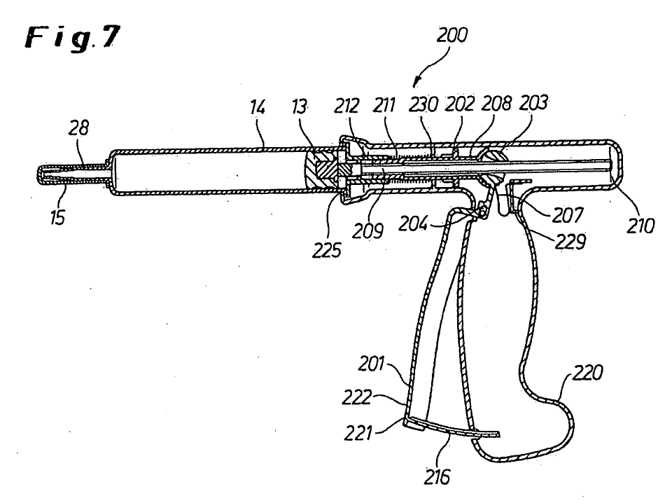

The lever 201 is used for the application of the apportioned mount and is mounted moveably at its proximal end 221 in the transition region between housing part 210 and the housing part 220 protruding therefrom. In FIG. 7, the distal end 222 of the lever 201 is spread apart from the protruding housing part 220, with the distance of the distal end 222 of the lever 201 from the housing part 220 being dependent on the dose set. The lever 201 is connected rotatably to the housing parts 210 and 220 at a fulcrum 204.

A scale 216 is fitted to the distal end 222 of the lever 201 and lies relative to the fulcrum 204 on the sector of the circular path of a circle, the radius of which corresponds to the distance between fulcrum 204 and the point 221 at which the scale 216 is fitted to the lever 201. The scale 216 is located to a greater or lesser extent within or outside the housing part 220, depending on the degree of spreading apart of the lever 201. At higher doses, a higher proportion of the scale 216 is located outside the housing part 220, and, correspondingly, at lower doses a smaller part. However, a scale may alternatively also be fitted directly to the dosing wheel 202 or at another suitable location.

The lever 201 has a ball 203 which is in contact with the dosing unit. The dosing unit contains a threaded spindle 209, a threaded sleeve 208, a dosing wheel 202, which is part of the threaded sleeve 208, and a latching mechanism for the threaded spindle 209.

The compression-spring dosing means 207 is mounted between a web 229, which is connected fixedly to the housing part 210 or 220, and the proximal end 221 of the lever 201 in such a manner that it exerts an axial pressure on the ball 203 in the direction of the front end of the housing part 210.

When the cartridge 14 is inserted, the latching mechanism for the threaded sleeve 202 permits only a displacement of the threaded sleeve 202 relative to the threaded spindle 209 in the axial direction to the rear end of the housing 210, but not in the opposite direction. When the cartridge 14 is inserted, the latching mechanism for the threaded spindle 9 permits only a displacement of the threaded spindle 9 in the direction to the front end of the housing 10, but not in the opposite direction. The latching mechanism for the threaded spindle 9 is connected to the adapter for the cartridge 14 in such a manner that, when the cartridge 14 is removed from the adapter, the latching mechanism for the threaded spindle 9 is simultaneously released and any desired displacement of the threaded spindle 9 in the axial direction relative to the housing 210 and to the threaded sleeve 208 is possible.

FIG. 7 shows a cartridge 14 which is fastened to the front end of the housing part 210 via the adapter.

To set the dose, the dosing wheel 202 and therefore the threaded sleeve 208 are rotated and therefore displaced axially to the rear end of the housing part 210 via the thread of the threaded spindle 209. The ball 203 is displaced with the threaded sleeve 208 likewise in the axial direction to the rear end of the housing part 210. By displacement of the ball 203 in the direction of the rear end of the housing part 210, the lever 201 pivots out via the fulcrum 204 and, with increasing axial displacement of the ball 203, spreads apart ever more from the housing part 220. The dose set can be gathered from the scale 216.

The threaded spindle 209 is prevented by the latching mechanism for the threaded spindle 209 from being displaced axially to the rear with the threaded sleeve 208.

For the application, the lever 201 is pressed in the direction of the housing part 220. In the process, the ball 203 is displaced in the axial direction to the front end of the housing part 210. The threaded sleeve 208 and the threaded spindle 209 move with the ball 203 axially in the direction of the front end of the housing part 210.

The threaded spindle 209 presses against the plunger 213 of the cartridge 14 which is fastened to the adapter at the front end of the housing part 210. The plunger 13 is therefore moved in the cartridge 14 and, for its part, presses the amount of medium corresponding to the dose set out of the cartridge 14 through the mouthpiece 28.

For another application, a desired dosing volume can be set again by the dosing wheel 203. After setting and application of a first dose and every further dose, each time the lever 201 is actuated the threaded spindle 209 is pushed further in the axial direction out of the front end of the housing part 210.

In the previously described embodiments of the invention, the advance of the threaded spindle 9, 209 is dependent on the travel of the ball upon a lever pressure, the stroke, and on the pitch of the quick-acting screw thread of the threaded sleeve 8, 208. Furthermore, the advance of the threaded spindle per lever pressure can be varied by a transmission means which is arranged concentrically between the threaded sleeve 8, 208 and the threaded spindle 9, 209.

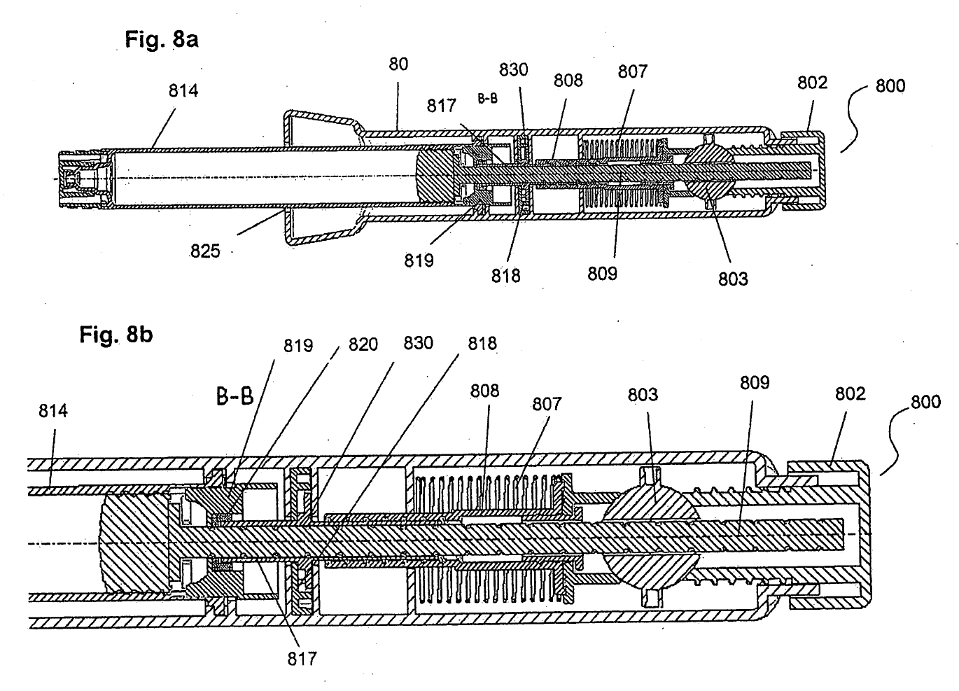

Such an embodiment with stroke transmission is described using the example of the applicator 800 from FIGS. 8a and 8b. In the case of the applicator 800 in FIGS. 8a and 8b, the adapter is not located on the passage opening 825 at the front end of the housing 80, but rather is located in the interior of the housing 80. A cartridge 814, which is fitted to the applicator 800, is therefore located partially within the housing 80. The dosing unit in the applicator 800 has a dosing wheel 802, a ball 803, a threaded spindle 809, a compression-spring dosing means 807 and a threaded sleeve 808 which interact in the manner described for the adapter 100 in FIGS. 1-4. The lever for the dosing operation is not shown for this embodiment.

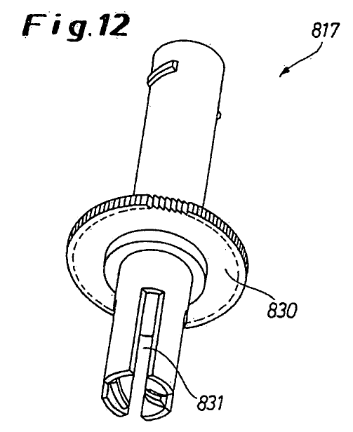

A slit transmission sleeve 817 (also see FIG. 12, shown here without a thread) with a thread located on the inside and a thread acting in the same direction (for example, both right-hand threads) located on the outside is mounted concentrically between the threaded sleeve 808 and the threaded spindle 809. The transmission sleeve 817 has an annular widening 830 in its central region. The transmission sleeve 817 is mounted rotatably in the housing 80. The transmission sleeve 817 interacts by means of its annular widening 830, which has a sawtooth-shaped structuring on its outer circumference, with a latching element 818 in such a manner that, for the transmission sleeve 817, only a rotation in a direction opposed to the thread direction (for example left rotation in the case of a right-hand thread on the inside and outside) is possible, but not in the reverse direction.

The internal thread of the transmission sleeve 817 has a different pitch from the internal thread of the threaded sleeve 808. The advance of the threaded spindle 809 changes in accordance with the ratio between pitch of the internal thread of the threaded sleeve 808 and pitch of the internal thread of the transmission sleeve 817.

If the internal thread of the threaded sleeve 808 (and also the external thread of the transmission sleeve 817) has, for example, a pitch of 5 mm, a stroke of 10 mm produces two revolutions of the transmission sleeve 817. If the internal thread of the transmission sleeve 817 (and the thread of the threaded spindle 809) has, for example, a pitch of 10 mm, then, with two revolutions of the transmission sleeve 817, the threaded spindle 809 is moved forward by 20 mm. A transmission ratio of the original stroke of 1:2 is produced therefrom.

So that the transmission sleeve 817 does not execute a return stroke when the threaded sleeve 808 is rotated back, the reverse movement is blocked by the latching element 818.

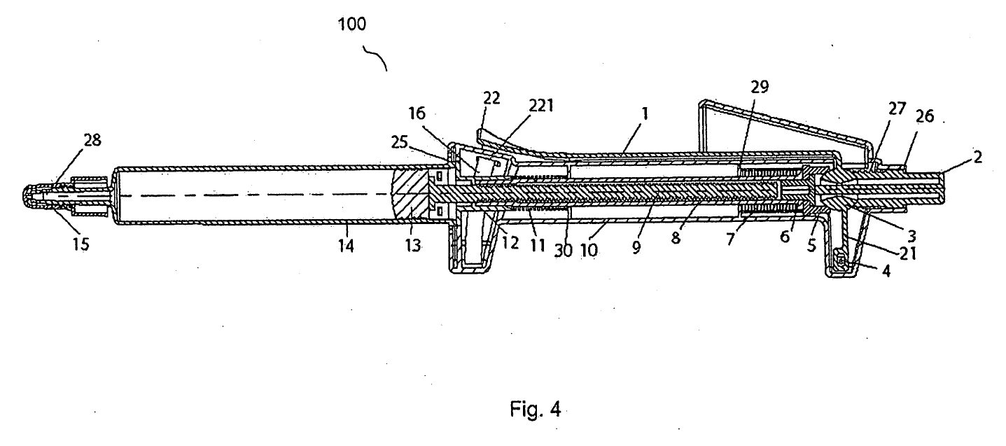

The adapter for the fastening of the cartridge 14 can be arranged on the passage opening 25 at the front end of the housing 10 (FIGS. 3a, 4) or else in the interior of the housing 80, as shown in FIGS. 8a, b. The functioning of the adapter is described below by way of example with reference to the embodiment of the applicator from FIG. 3a.

The adapter in the applicator 100 has a compression-spring closure 11 and a lock nut 12. The lock nut 12 is a tubular sleeve which is mounted displaceably in the axial direction concentrically with the threaded spindle 9 and directly behind the passage opening 25. The passage opening 25 has a circular shape which is interrupted at two mutually opposite locations, the additional openings 39, and has a somewhat larger diameter at these additional openings 39 than in the circular region (FIG. 3b).

At its front end facing the passage opening 25, the lock nut 12 has an annular widening, the diameter of which is greater than the diameter of the circular passage opening 25.

The lock nut 12 has, on its inner wall, an annular thickening, the clear opening of which corresponds to the diameter of the threaded spindle 9.

In the state ready for use, i.e. when a cartridge 14 is fastened to the adapter, the front end of the threaded sleeve 8 is located within the tubular sleeve of the lock nut 12. The annular thickening on the inner wall of the lock nut 12 stops an axial displacement of the threaded sleeve 8 in the direction of the front end of the housing 10.

The compression-spring closure 11 is mounted between a ring 30, which is connected fixedly to the housing 10, and the rear end of the lock nut 12 in such a manner that it exerts axial pressure on the rear end of the lock nut 12 in the direction of the front end of the housing 10.

If there is not a cartridge on the adapter, then the lock nut 12 is displaced by the compression-spring closure 11 toward the front end of the housing 10 until the front end of the threaded sleeve 8 is no longer located within the tubular sleeve of the lock nut 12. The threaded sleeve 8 is slit longitudinally such that it spreads as soon as its front end is no longer held together by the tubular part of the lock nut 12. This spreading is sufficient in order to undo the close contact between threaded spindle 9 and threaded sleeve 8 such that the threaded spindle 9 can then be moved in the axial direction through the threaded sleeve 8 without carrying along the threaded sleeve 8.

The cartridge 14 (FIG. 3a, FIG. 9) has a cylindrical wall 21. That end of the cartridge 14 which faces the applicator 100 has an opening 31 for filling the cartridge 14, which opening is closed by a plunger 13 in the state ready for use. The other end of the cartridge 14 tapers to form a mouthpiece 28 which is closed by a closure cap 15. In the region of the opening 31, the wall 21 of the cartridge 14 is widened to form a ring 33. The diameter of the ring 33 corresponds to the diameter of the passage opening 25 of the applicator 100. At two mutually opposite locations, the ring 33 is widened by the extension pieces 34. The diameter of the cartridge 14 in the region of the extension pieces 34 corresponds to the inside diameter of the additional openings 39 in the passage opening 25 (see FIG. 3b).

The cartridge 14 is fastened to the applicator 100 by being guided with the opening 31 onto the passage opening 25 such that the extension pieces 34 enter the interior of the housing 10 through the additional openings 39. In the process, axial pressure is exerted on the compression-spring closure 11 in the direction of the rear end of the housing 10 and the cartridge 14 is simultaneously rotated through 90 degrees, with the result that the extension pieces 34 come to lie behind the circular passage opening 25 and are clamped or latched into place there.

The cartridge 14 is normally removed from the applicator 100 when its content has been consumed, i.e. when the threaded spindle 9 has been pushed to a maximum extent out of the front end of the housing 10. In this case, it is rotated through 90 degrees under slight pressure against the compression-spring closure 11 such that the latch-in position is overcome and the extension pieces 34 come free in the additional openings 39. Before a further cartridge 14 is fitted, the threaded spindle 9 has to be pushed back again into its initial position in the housing 10. This is possible by the threaded sleeve 8 being slit longitudinally. As long as a cartridge 14 is located on the applicator 100, the longitudinally slit threaded sleeve 8 is held together by the lock nut 12. If the lock nut 12 is released because the cartridge 14 is removed from the adapter, the threaded sleeve spreads slightly and the threaded spindle 9 can be pushed back without rotation under axial pressure.

A variant of the adapter is described with reference to the embodiment of the applicator according to FIGS. 8 a, b. However, the variant may also analogously be combined with the embodiment of the applicator according to FIG. 3a. In a departure from the above-described embodiment, in the variant the locking of the cartridge 814 does not take place with the aid of a spring but rather by means of a lock nut element 819. In the case of the applicator 800 according to FIG. 8a, the passage opening 825 is designed analogously to that in the case of the applicator 100 according to FIG. 3, in circular form with additional openings 39 (see FIG. 3b).

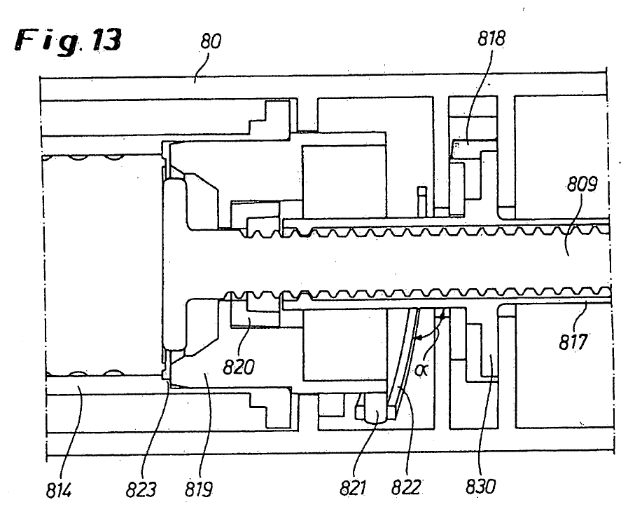

In this variant, the adapter on the applicator 800 (also see FIG. 13) has a lock nut element 819 and a bushing 820. The lock nut element 819 is mounted rotatably in the housing 80. The lock nut element 819 is a two-part, tubular sleeve which is mounted displaceably in the axial direction concentrically with the threaded spindle 809 and with the transmission sleeve 817. On its side facing the passage opening 825, it has, on its outer circumference, grooves which extend in the axial direction and to which a correspondingly adapted cartridge 814 can be coupled. On its side facing away from the passage opening 825, it has, on its outer circumference, a cam 821 which engages in a channel 822 which is provided on the inner wall of the housing 80. The channel 822 runs on the inner wall of the housing 80 at an angle α to the axial direction, with α, as shown in FIG. 13, lying between 90 and 100 degrees, preferably between 90.5 and 95 degrees.

Upon a rotation of the lock nut element 819, the lock nut element 819 is simultaneously displaced in the axial direction, guided by the cam 821 in the channel 822. A bushing 820 with a central hole through which the threaded spindle 809 fits is located centrally in the interior of the lock nut element. Upon axial displacement of the lock nut element, the bushing 820 is pushed in a direction away from the passage opening 825 over that end of the transmission sleeve 817 which faces the passage opening 825. Since the transmission sleeve (also see FIG. 12) is designed elastically by means of slits 831 and spreads, the displacement of the bushing 820 over the end of the transmission sleeve 817 produces the interlocking connection between the threaded spindle 809 and the transmission sleeve 817.

The cartridge 814 (see, for example, FIGS. 8a, 13) differs from the cartridge 14 (FIG. 9) in that, in its inner circumference, in the region of the opening 31, it has one or more elongate projections 823 extending in the axial direction. The position of these elongate projections 823 is selected in such a manner that they coincide with the position of the grooves in the outer circumference of the lock nut element 819 (see FIG. 13).

When the cartridge 814 is fitted, the cartridge 814 is rotated such that the positions of the extension pieces 34 coincide with the position of the additional openings 39 in the passage opening 825. This means that the position of the grooves on the lock nut element 819 and the projections 862 on the inner wall of the cartridge 814 coincide at the same time. Thus positioned, the cartridge 814 is pushed over the lock nut element 819. With a rotational movement of the cartridge 814 through, for example, 90 degrees, the lock nut element 819 executes an axial movement relative to the housing 80 via the interaction of cam 921 and channel 822. During this movement, the transmission sleeve 817 is enclosed and the interlocking connection with the thread of the threaded spindle 809 produced. The bushing 820 also prevents lateral forces from acting on the bearing of the transmission sleeve 817.

In their final position, the extension pieces 34 latch into depressions or apertures on the inner wall of the housing 80.

The cartridge 814 is normally removed from the applicator 800 when its content has been consumed, i.e. when the threaded spindle 809 has been pushed to a maximum extent out of the front end of the housing 80. To remove it, the cartridge 814 is rotated with slight pressure until the latch-in position of the extension pieces 934 on the housing 80 is overcome and the lock nut element 819 has moved back in the axial direction onto the passage opening 825 into the initial position prior to the fitting of the cartridge 814.

Before a further cartridge 814 is fitted, the threaded spindle 809 has to be pushed back again into its initial position in the housing 80. As long as a cartridge 814 is fastened in the applicator 800, the longitudinally slit transmission sleeve 817 is held together by the bushing 820. If the lock nut element 819 is moved axially, however, in the direction of the passage opening 825, the bushing 820 at some point no longer engages around the end of the transmission sleeve 817, and the transmission sleeve 817 spreads slightly and the threaded spindle 809 can be pushed back without rotation under axial pressure.

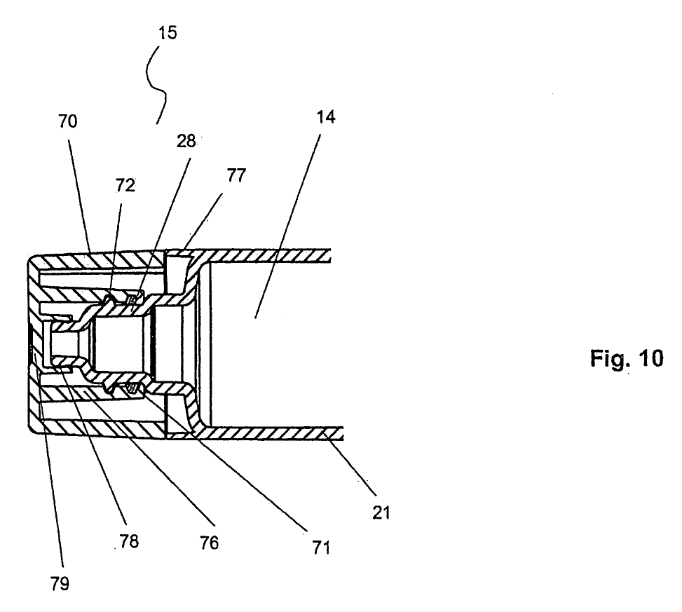

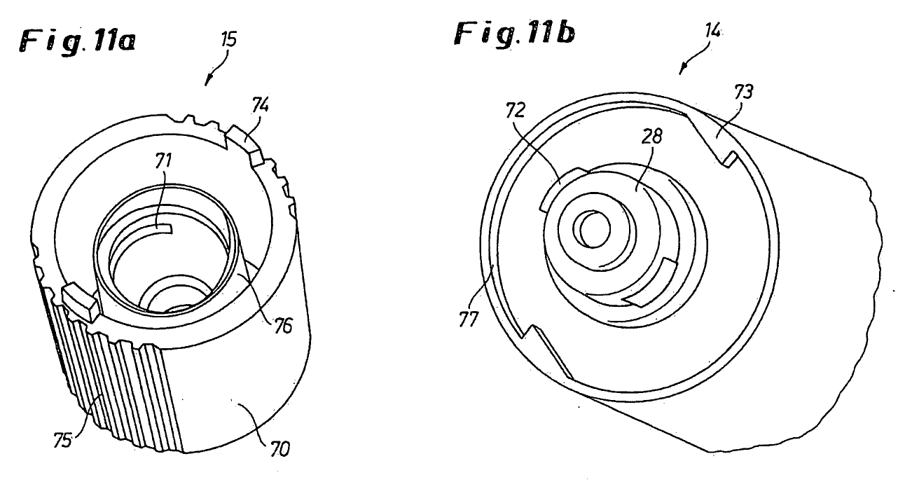

FIGS. 10 and 11a show a closure cap 15 with a child-proof lock for closing the mouthpiece 28 of the cartridge 14, 814. The closure cap 15 has an outer cylindrical wall 70 and an end wall 79. The outside diameter of the closure cap 15 corresponds to the outside diameter of the cartridge 14. An inner cylindrical wall 76 is located concentrically within the outer wall 70. A thread 71 is located on the inside of the inner wall 76. On the open side of the closure cap 15, two mutually opposite cams 74 are located on the end surface of the outer wall 70 and at a small distance from the outside of the outer wall 70. On the outside of the outer wall 70, structures 75 are incorporated in the region of the cam 74 and permit secure gripping and firm holding of the closure cap 15 in these regions.

The mouthpiece 28 of the cartridge 14, 814 has threaded wings 72 (FIGS. 10, 11b) which are arranged in such a manner that they engage in the thread 71 when the closure cap 15 is screwed to the mouthpiece 28. In that region of the cartridge 14 in which it tapers to the mouthpiece 28, the wall 21 forms an annular edge 77, on the inside of which two mutually opposite, sawtooth-shaped projections 73 are located. When the closure cap 15 is screwed to the cartridge 14, the cams 74 are guided past the projections 73 and latch behind them such that rotation back is no longer possible. To rotate it back, i.e. to unscrew the closure cap, the latching of the cams 74 on the projections 73 has to be overcome. This takes place by the outer wall 70 of the closure cap 25 being compressed in the region of the cams 74 until the cams 74 can be guided on the inside past the projections 73.

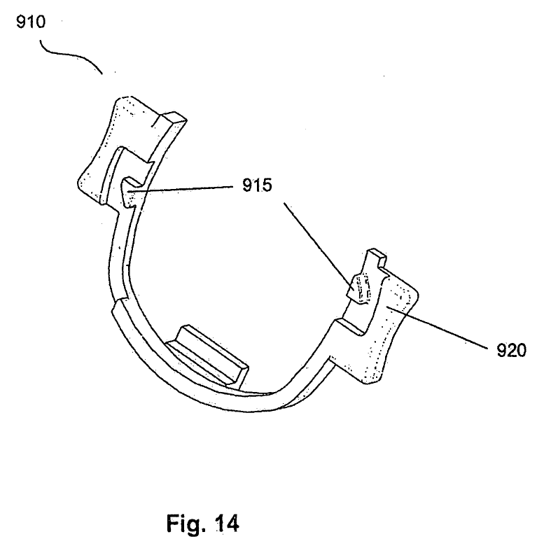

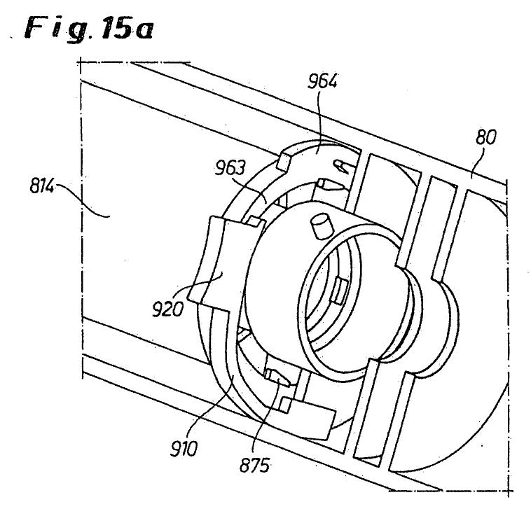

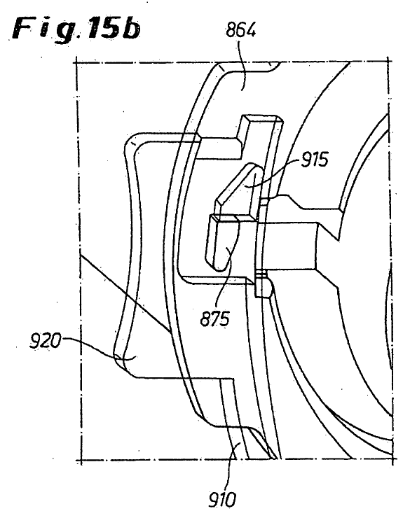

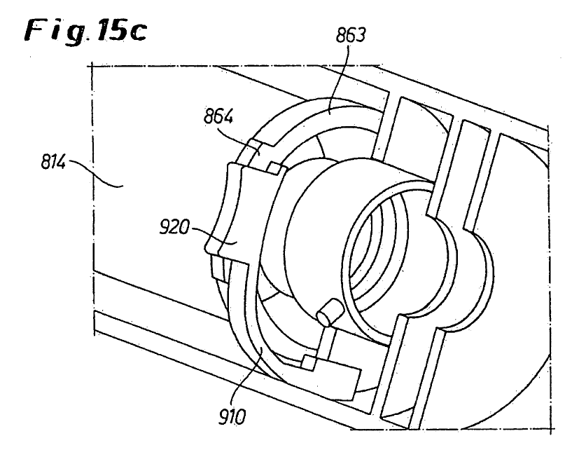

In a preferred embodiment of the applicator 100 or 800, the adapter for fastening the cartridge 14 or 814 additionally comprises an approximately semicircular latching arc 910 for locking the cartridge 14, 814. The latching arc (FIGS. 14, 15a-c) is mounted concentrically in the housing 10, 80 and has latching cams 915 which latch with corresponding latching cams 875 on the extension pieces 34 on the cartridge 14, 814.

The function of the latching arc 910 in conjunction with the applicator 800 in the embodiment according to FIGS. 8a, b and with a cartridge 814 is described below. It goes without saying that the function of the latching arc 910 can be implemented in precisely the same manner in conjunction with the other applicator embodiments described herein.

FIG. 14 shows the approximately semicircular latching arc 910 with the two sensing devices 920 arranged opposite each other. By means of the arcuate form, the latching arc 910 is elastic and can be deformed by radial pressure from the outside on the sensing devices 920 in such a manner that the sensing devices 920 come closer to each other. After the sensing devices 920 are released, the latching arc 910 resumes its original shape. In the region of the sensing devices 920, sawtooth-shaped latching cams 920, which both point with their tips in the same direction of rotation, are arranged on the inside of the latching arc 910.

FIGS. 15a-c show how the latching arc 910 is arranged in the housing 80 of the applicator 800. In this case, the cartridge 814 is shown in the unlatched position in FIG. 15a and in the latched position in FIG. 15c. The sensing devices 920 are arranged in apertures through the housing 80 of the applicator 800 in such a manner that they can be operated, i.e. pressed, from outside the housing.

In the latched state (also see FIG. 15b), the sawtooth-shaped latching cams 875 on the extension pieces 39 of the cartridge 814 engage in the sawtooth-shaped latching cams 915 of the latching arc 910 in such a manner that rotation of the cartridge 814 back is no longer possible. Only by pressing on both sensing devices 920 simultaneously is the latching of the respective latching cams 975 and 915 at the two mutually opposite positions released. This takes place by the sensing devices 920 being compressed until the latching cams 915 of the latching arc 910 are displaced into the interior of the housing 800 until the latching cams 875 of the cartridge 814 can be guided on the inside past the latching cams 915 of the latching arc 910.

1. An applicator (100) for the application of liquid or pasty media, containing
a housing (10) for receiving a dosing unit and an adapter for fastening a container (14), for the medium to be applied, to the applicator (100);
a dose setting means (2);
a lever (1) for the application of the apportioned amount, the lever (1) interacting mechanically with the dosing unit and with the dose setting means (2),
characterized in that
the lever (1) is mounted moveably at its proximal end within the housing (10) and in that the distal end of the lever (1) is spread apart from the housing (10) in the state ready for application, with the distance of the distal end of the lever (1) from the housing (10) being dependent on the dose set.

2. The applicator (100) as claimed in claim 1, characterized in that the dose setting means (2) is a dosing wheel.

3. The applicator (100) as claimed in claim 1, characterized in that the lever (1) is connected rotatably to the housing (10) at a fulcrum.

4. The applicator (100) as claimed in claim 1, characterized in that the lever (1) has a ball (3) which is in contact with the dose setting, means (2) and the dosing unit.

5. The applicator (100) as claimed in claim 1, characterized in that a scale (16) is fitted to the distal end of the lever (1) or to the housing (10) in such a manner that a larger or smaller part of the scale (16) is visible, depending on the degree of spreading apart of the lever (1).

6. The applicator (100) as claimed in claim 5, characterized in that a scale (16) is fitted to the dose setting means (2).

7. The applicator (100) as claimed in claim 1, characterized in that the lever (1) is latched to the housing (10).

8. The applicator (100) as claimed in claim 1, characterized in that the dosing unit has a threaded spindle (9), a threaded sleeve (8), a pressure-exerting means (7), a latching mechanism for the threaded spindle (9) and a latching mechanism for the threaded sleeve (8) and these elements interact with the lever (11) in such a manner that the threaded sleeve (8) is displaced relative to the threaded spindle (9) by the axial force of the pressure-exerting means (7) during setting of the dose and, each time the lever (1) is actuated, the threaded spindle (9) is pushed further axially in a direction out of the threaded sleeve (8).

9. The applicator (100) as claimed in claim 8, characterized in that it has a latching mechanism for the threaded sleeve (8), which latching mechanism, when the container (14) for the medium to be applied is inserted in the adapter, permits a displacement of the threaded sleeve (8) only in an axial direction relative to the threaded spindle (9).

10. The applicator (100) as claimed in claim 8, characterized in that it has a latching mechanism for the threaded spindle (9), which latching mechanism, when the container (14) for the medium to be applied is inserted in the adapter, permits a displacement of the threaded spindle (9) only in an axial direction which is opposed to the axial direction in which the latching mechanism for the threaded sleeve (8) permits a displacement.

11. The applicator (100) as claimed in claim 8, characterized in that the pressure-exerting means (7) is a compression spring.

12. The applicator (100, 800) as claimed in claim 8, characterized in that a transmission sleeve (817) is arranged concentrically between the threaded sleeve (8, 808) and the threaded spindle (9, 809).

13. The applicator (100, 800) as claimed in claim 8, characterized in that the threaded spindle (9, 809) has a quick-acting screw thread.

14. The applicator (100, 800) as claimed in claim 8, characterized in that the threaded sleeve (8, 808) or the transmission sleeve (817) is partially slit longitudinally.

15. The applicator (100, 800) as claimed in claim 1, characterized in that the housing (10) has a passage opening (25) for the container (14, 814) in the region of the adapter.

16. The applicator (100, 800) as claimed in claim 15, characterized in that the passage opening (25) for the container (14, 814) is circular and has two to five, preferably two mutually opposite additional openings (39).

17. The applicator (100) as claimed in claim 1, characterized in that the adapter has a lock nut (12) and a pressure-exerting means (11), the lock nut (12) being mounted concentrically with the threaded spindle (9) and the pressure-exerting means (11) pressing the lock nut (12) in the direction of the passage opening (25) for the container (14).

18. The applicator (100) as claimed in claim 17, characterized in that the lock nut (12) is mounted in such a manner that it engages around that end of the threaded sleeve (9) or of the transmission sleeve (817) which faces the passage opening (25) only when the container (14) is inserted in the adapter.

19. The applicator (100, 800) as claimed in claim 1, characterized in that the adapter has a lock nut element (819) which is concentric with the threaded spindle (9, 809), has one or more grooves or projections extending in the axial direction on its outer circumference and is mounted in such a manner that a rotational movement of the lock nut element (819) results simultaneously in an axial displacement of the lock nut element (819).

20. The applicator (100, 800) as claimed in claim 19, characterized in that the lock nut element (819) is mounted in such a manner that it engages around that end of the threaded sleeve (8, 808) or of the transmission sleeve (817) which faces the passage opening (25) only when the container (14, 814) is inserted in the adapter.

21. The applicator (100, 800) as claimed in claim 1, characterized in that the applicator (100) has a latching arc (910) with two sensing devices (920), which latching arc locks a container (14, 814) inserted into the adapter and said locking can be released only by simultaneous pressing of the two sensing devices (920).

22. A container (14, 814) for receiving a liquid or pasty medium, having a cylindrical housing (21) with a filling opening (31) on the one side and with a tapered region (28) on the other side of the housing (21), the tapered region (28) having an opening for dispensing the liquid or pasty medium, characterized in that the container (14, 814) is suitable for being fastened to an applicator (100, 800) as claimed in one of claims 1 to 21 with the aid of the adapter.

23. The container (14, 814) as claimed in claim 22, characterized in that the container (14, 814) has extension pieces (34) in the region of the edge around the filling opening (31), the number, size and position of the extension pieces (34) coinciding with the number, size and position of the additional openings (39) in the passage opening (25).

24. The container (14, 814) as claimed in claim 22, characterized in that the inner wall of the container (14, 814) has grooves or projections (823) oriented in the axial direction in the region of the filling opening (31), the number and position of which grooves or projections coincides with the number and position of the grooves or projections in the outer circumference of the lock nut element (819).

25. The container (14, 814) as claimed in claim 22, characterized in that it has a volume within the range of from 5 ml to 50 ml, preferably within the range from 8 ml to 20 ml, particularly preferably from 11 to 13 ml.

26. The container (14, 814) as claimed in claim 22, characterized in that a closure cap (15) is fitted on the tapered region (28).

27. The container (14, 814) as claimed in claim 26, characterized in that the closure cap (15) has a child-proof lock.

28. The container (14, 814) as claimed in claim 22, characterized in that it is filled with a liquid or pasty medium.

29. The container (14, 814) as claimed in claim 28, characterized in that the liquid or pasty medium is an animal drug.

30. An applicator system, containing an applicator (100, 800) as claimed in claim 1, to which a container (14, 814) as claimed in claim 22 is fastened.

31. The applicator system as claimed in claim 30, characterized in that the container (14, 814) is fitted to the housing (10) with the aid of the adapter in such a manner that, upon a lever pressure, the threaded spindle (9, 809) moves into the container (14, 814) coaxially therewith.

32. The applicator system as claimed in claim 30, characterized in that each lever pressure corresponds to a dosing volume within the range of from 0.1 to 2.5 ml.

33.-34. (canceled)
